(19) United States
(12) Patent Application Publication
(10) Pub. No.: US 2019/0216383 A1
(43) Pub. Date: Jul. 18, 2019

(54) COGNITIVE SUPPORT SYSTEM FOR A SUFFERER OF COGNITIVE DYSFUNCTION AND METHOD FOR OPERATING THE SAME

(71) Applicant: Robert Polkowski, Boston, MA (US)

(72) Inventor: Robert Polkowski, Boston, MA (US)

(21) Appl. No.: 16/362,299

(22) Filed: Mar. 22, 2019

Related U.S. Application Data
(63) Continuation-in-part of application No. 15/196,943, filed on Jun. 29, 2016.

(51) Int. Cl. A61B 5/00; A61B 5/11; A61B 5/053; A61B 5/024; A61B 5/16; A61B 5/1172

(52) U.S. Cl. CPC A61B 5/4088; A61B 5/0002; A61B 5/1118; A61B 5/0533; A61B 5/02055; A61B 5/165; A61B 5/1172; A61B 5/1116; A61B 5/02438

(57) ABSTRACT

A cognitive support system is disclosed. The system includes a data collection subsystem to obtain personal data and contextual data related to the sufferer, from conversations of one or more family members, one or more close friends and one or more co-workers. The system includes a cognitive modelling subsystem to analyse the personal data and the contextual data to build a personalized support model. The system includes a cognitive support subsystem to learn one or more patterns from the personal data and the contextual data based on the personalized support model, verify one or more learnt patterns based on the personal data and the contextual data gathered from the conversations, identify one or more instances of cognitive dysfunction based on the one or more learnt patterns and a current personal data of the sufferer and generate an intervention for the sufferer upon identifying one or more instances of cognitive dysfunction.

### CROSS-REFERENCE TO RELATED APPLICATION

This application is a continuation-in-part of, and claims the priority benefit of, pending U.S. Non-Provisional Application bearing application No. 15/196,943, filed on Jun. 29, 2016, entitled “Artificial Intelligence Support for Sufferers of Cognitive Dysfunction,” which is hereby incorporated by reference in its entirety.

### BACKGROUND

Embodiments of a present disclosure relate to artificial cognition and more particularly to cognitive support system for a sufferer of cognitive dysfunction and method for operating the same.

Cognitive dysfunction is a category of mental health disorders which primarily affects cognitive abilities including learning, memory, perception, and problem solving. The disorders usually advance gradually, affecting different areas of the brain at an increasing rate. A sufferer affected by cognitive dysfunction may be prescribed medications or adopt lifestyle changes in order to alter the course of their condition. However, such methods are known to slow the progress or alleviate the symptoms of the underlying disease, but not to fully stop the progression or provide a cure. As a result, once the severity of the conditions increases, family members or nursing professionals are required for constant supervision and care.

Various systems have been introduced which helps the sufferer of cognitive dysfunction in performing the activities of the daily living. One such system includes scheduling and planning tools which may not be helpful in such situations, because an individual may need a reminder to use the tools themselves, and the tools may require significant preparation maintenance. Moreover, such tools are not helpful in dynamically changing situations when a person needs to be reminded to take an action that depends on a previous action or sequence of actions. Also, such tools cannot assist in more complex use cases such as an individual to maintain a conversation in real-time when said person experiences difficulty with expressive communication (i.e. inability to recall familiar words to express a concept). Such approaches suffer from a poor ability to recognize the context of the sufferer and deploy assistive aids in an adaptive fashion.

Furthermore, some of the existing systems identify the instances of cognitive dysfunction and generates an intervention to assist the sufferer after collecting personal data and historic data related to the sufferer. However, such systems are not confident enough to prompt the sufferer without verifying the information with the related individuals or the caregiver. Also, such systems identify the patterns from the personal data and the historic data and suggests the activity from the same pattern without verifying the correctness of the pattern, thereby result in opt-in fashion (with permission granted by the user, caregiver, or guardian) response.

Hence, there is a need for an improved cognitive support system for a sufferer of cognitive dysfunction to address the aforementioned issue(s).

### SUMMARY

In accordance with an embodiment of the present disclosure, a cognitive support system for a sufferer of cognitive dysfunction is provided. The system includes a data collection subsystem located on a remote server. The data collection subsystem is configured to gather personal data and contextual data, related to the sufferer, from conversations of one or more family members, one or more close friends and one or more co-workers of the sufferer. The system also includes a cognitive modelling subsystem operatively coupled to the data collection subsystem. The cognitive modelling subsystem is configured to analyse the personal data and the contextual data related to the sufferer of cognitive dysfunction. The cognitive modelling subsystem is also configured to build a personalized support model based on an analysed data. The system further includes a cognitive support subsystem operatively coupled to the cognitive modelling subsystem. The cognitive support subsystem is configured to learn one or more patterns from the personal data and the contextual data related to the sufferer of the cognitive dysfunction based on the personalized support model. The cognitive support subsystem is also configured to verify one or more learnt patterns based on the personal data and the contextual data gathered from the conversations of the one or more family members, the one or more close friends and the one or more co-workers of the sufferer. The cognitive support subsystem is also configured to identify one or more instances of cognitive dysfunction based on the one or more learnt patterns and a current personal data related to the sufferer of the cognitive dysfunction obtained from a monitoring device. The cognitive support subsystem is further configured to generate an intervention for the sufferer upon identifying one or more instances of cognitive dysfunction based on the personalized support model.

In accordance with another embodiment of the present disclosure, a method for operating the cognitive support system is provided. The method includes gathering, by a data collection subsystem, personal data and contextual data, related to the sufferer, from conversations of one or more family members, one or more close friends and one or more co-workers of the sufferer. The method also includes analysing, by a cognitive modelling subsystem, the personal data and the contextual data related to the sufferer of cognitive dysfunction. The method further includes building, by the cognitive modelling subsystem, a personalized support model based on an analysed data. The method further includes learning, by a cognitive support system, one or more patterns from the personal data and the contextual data related to the sufferer of the cognitive dysfunction based on the personalized support model. The method also includes verifying, by the cognitive support subsystem, one or more learnt patterns based on the personal data and the contextual data gathered from the conversations of the one or more family members, the one or more close friends and the one or more co-workers of the sufferer. The method further includes identifying, by the cognitive support system, one or more instances of cognitive dysfunction based on the one or more learnt patterns and a current personal data related to the sufferer of the cognitive dysfunction obtained from a monitoring device. The method further includes generating, by the cognitive support system, an intervention for the sufferer upon identifying one or more instances of cognitive dysfunction based on the personalized support model.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a cognitive support system for a sufferer of cognitive dysfunction in accordance with an embodiment of the present disclosure;

FIG. 2 is a block diagram representation of an exemplary cognitive support system for the sufferer of cognitive dysfunction of FIG. 1 in accordance with an embodiment of the present disclosure; and

FIG. 3 is a flow chart representing the steps involved in a method for operating the cognitive support system of FIG. 1 in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a cognitive support system for a sufferer of cognitive dysfunction. The system includes a data collection subsystem located on a remote server. The data collection subsystem is configured to gather personal data and contextual data, related to the sufferer, from conversations of one or more family members, one or more close friends and one or more co-workers of the sufferer. The system also includes a cognitive modelling subsystem operatively coupled to the data collection subsystem. The cognitive modelling subsystem is configured to analyse the personal data and the contextual data related to the sufferer of cognitive dysfunction. The cognitive modelling subsystem is also configured to build a personalized support model based on an analysed data. The system further includes a cognitive support subsystem operatively coupled to the cognitive modelling subsystem. The cognitive support subsystem is configured to learn one or more patterns from the personal data and the contextual data related to the sufferer of the cognitive dysfunction based on the personalized support model. The cognitive support subsystem is also configured to verify one or more learnt patterns based on the personal data and the contextual data gathered from the conversations of the one or more family members, the one or more close friends and the one or more co-workers of the sufferer. The cognitive support subsystem is also configured to identify one or more instances of cognitive dysfunction based on the one or more learnt patterns and a current personal data related to the sufferer of the cognitive dysfunction obtained from a monitoring device. The cognitive support subsystem is further configured to generate an intervention for the sufferer upon identifying one or more instances of cognitive dysfunction based on the personalized support model.

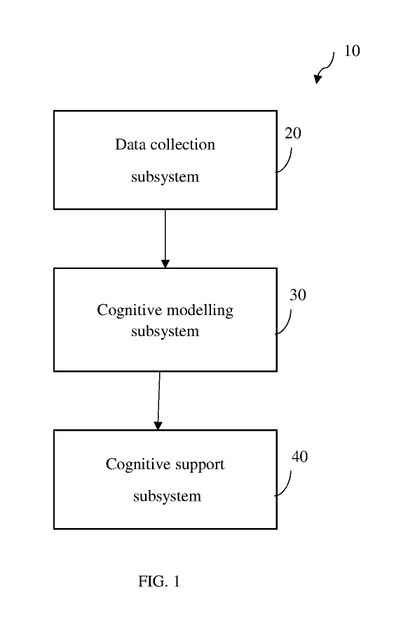

FIG. 1 is a block diagram representation of a cognitive support system 10 for a sufferer of cognitive dysfunction in accordance with an embodiment of the present disclosure. As used herein, “cognitive dysfunction” is defined as a category of mental health disorders that primarily affects cognitive abilities including learning, memory, perception, and problem solving of a person. The cognitive support system 10 is configured to assist a sufferer of cognitive dysfunction by providing real time assistance when the sufferer is unable to perform a desired task. The cognitive support system 10 includes a data collection subsystem 20 which is located on a remote server (not shown in FIG. 1). The data collection subsystem 20 is configured to gather personal data and contextual data, related to the sufferer, from conversations of one or more family members, one or more close friends and one or more co-workers of the sufferer. In one embodiment, the conversations of one or more family members, one or more close friends and one or more co-workers may include telephonic conversations. In another embodiment, conversations of one or more family members, one or more close friends and one or more co-workers may include non-telephonic face-to-face conversation. In a specific embodiment, the data collection subsystem 20 may gather data from the individuals in surrounding of the sufferer which can be family member, relative, friend, co-worker or any other unknown or known person (such as shopkeeper, maid, or watchman). In one embodiment, the personal data may include a historical data of the sufferer and a present situation data of the sufferer. In such embodiment, the historical data of the sufferer and the present situation data of the sufferer may include a heart rate, skin conductance response, skin temperature, posture, motion, voice, reaction to outer stimuli, internal and external location, orientation data, sound, data corresponding to ambient light level, data corresponding to ambient geomagnetic field and proximity data, and environment data based on IoT sensors coupled to a plurality of objects in the home. In some embodiments, the personal data may include digital footprint including at least one of social media profiles, documents, electronic mails and recordings.

In one embodiment, the contextual data may include data about the preferences and background of the user, wherein the data about the preferences and background of the user may include daily living activities, family composition, social situations, professional history, medical history and entertainment preferences and data related to spanning the lifetime of the sufferer. In such embodiment, the daily living activities may include daily routine such as get up, have breakfast, go to work, have lunch, go back to home, have dinner or go to bed and the like. In some embodiment, the family composition may include family relations and family history data. In a specific embodiment, the social situations may include past chronological events related to sufferer and the associated time, date, conversations, people involved in the past events, and events occurred in life of the one or more family members, the one or more close friends and the one or more co-workers during the life span of the sufferer. In one embodiment, the professional history may include one or more work places of the sufferer, past activities at work places, meetings, interactions, interviews at work place and associated conversations and individuals related to sufferer and the like. In some embodiments, the medical history comprises family medical history of the sufferer (parents or grand patents related diseases), sufferer medical history (a plurality of visits to doctor and corresponding diseases and conversation with the doctor on each visit), data related to medicines which the sufferer is taking in past or in present and the like. In a specific embodiment, the entertainment preferences may include at least one of likes/dislikes, shopping habits, hobbies and the like. In one embodiment, the data related to spanning the life time of the sufferer may include important events such as marriage, birth of children, job placement, data associated with life events of the one or more family members that had happened during the life time of the sufferer; retirement and places of living during a span of the lifetime and the conversation associated with the important events and places of living.

Furthermore, the cognitive support system 10 includes a cognitive modelling subsystem 30 which is operatively coupled to the data collection subsystem 20. The cognitive modelling subsystem 30 is configured to analyse the personal data and the contextual data related to the sufferer of cognitive dysfunction. The cognitive modelling subsystem 30 is also configured to build a personalized support model based on an analysed data. The personalized support model may include a combination of one or more deep recurrent neural networks models and one or more convolutional neural networks models. In one embodiment, the personalized support model includes a business rule model which is configured to define, deploy, execute, monitor and maintain the variety and complexity of decision logic of selecting a suitable model or a combination of models as per one or more instances of cognitive dysfunction. In such embodiment, the personalized support model is built by domain knowledge regarding how the symptoms tend to affect individuals in real-life situations. In one embodiment, the personalized support model may be trained by providing individual's input to the personalized support model. In another embodiment, the personalized support model may be updated by detecting a plurality of symptoms in the sufferer's activities. In yet another embodiment, the personalized support model may be updated for identifying one or more exceptions that may require intervention.

Moreover, the system 10 further includes a cognitive support subsystem 40 which is operatively coupled to the cognitive modelling subsystem 30. The cognitive support subsystem 40 is configured to learn one or more patterns from the personal data and the contextual data related to the sufferer of the cognitive dysfunction based on the personalized support model. In one embodiment, the one or more patterns may include at least one of a pattern of repetition, a pattern of omission, or a pattern of abandonment. The cognitive support subsystem 40 is also configured to verify one or more learnt patterns based on the personal data and the contextual data gathered from the conversations of the one or more family members, the one or more close friends and the one or more co-workers of the sufferer. The cognitive support subsystem 40 is further configured to identify one or more instances of cognitive dysfunction based on the one or more learnt patterns and a current personal data related to the sufferer of the cognitive dysfunction obtained from a monitoring device. In some embodiments, the monitoring device (not shown in FIG. 1) may include a wearable device. In such embodiment, the wearable device may include a wrist watch, a strap or a wearable cloth. The one or more instances of cognitive dysfunction may include forgetfulness, inability to complete task or change in emotional state. For example, under certain conditions, patterns of forgetfulness may be identified by the tendency to repeat certain actions, such as running the same errand multiple times. Under a different set of conditions, patterns of forgetfulness may also be identified by a symptomatic inability to complete tasks, such as abandoning the errand halfway through completion.

The cognitive support subsystem 40 is further configured to generate an intervention for the sufferer upon identifying one or more instances of cognitive dysfunction based on the personalized support model. In one embodiment, the intervention may include one or more notifications. In such embodiment, the one or more notification may include at least one of a notification of context awareness, a notification with one or more instructions to carry out a task and a notification for assistance in speaking. The notification of context awareness may be for example, a notification to remind names of sufferer's relatives or some context-specific information from a particular conversation. The context-specific information from a particular conversation may be determined using a speech recognition technique, where the current speech pattern and word choice of the sufferer are analysed during the conversation to predict words and/or phrases that the sufferer likely intends to say but may not be able to remember. The notification with one or more instructions to carry out a task may be for example, a notification with instruction to pick up a key before leaving the house or turn on and off appliances at home after use. The one or more notifications for assistance in speaking may include a speech output, delivered to the sufferer and the one or more individuals in surrounding of the sufferer, to complete the sentence of the sufferer in a conversation with the one or more individuals in surrounding of the sufferer. In some embodiments, the one or more notifications may include at least one of a text notification, an audio notification, a graphical notification and a video notification. In one embodiment, the one or more notifications of intervention may be provided to a caregiver. As used herein, “caregiver” is any human other than the sufferer which is in the sufferer's environment or interacts with the sufferer for assistance or help. Thus, a “caregiver” in accordance with the present invention is not limited to a medical specialist, a relative, a neighbour or a guest.

In one embodiment, a plurality of large-scale data patterns also contributes to the personalized support model. If we see a pattern commonly takes place under a similar set of circumstances for a cohort of sufferer, the system (10) add that pattern to a sufferer's personalized support model as a precaution. In a scenario for example, if the system (10) notices a pattern where certain groups of sufferers who forget their destination while driving tend to get into car accidents within three months of the first occurrence, the system (10) configures the personalized support model for the sufferers in those groups to target the patterns that may lead to car accidents for that cohort (if based on the plurality of large scale data patterns learnings, answering the phone while driving significantly increases the risk of accidents for sufferers in that cohort, that learning is integrated into personalized support models for those sufferers).

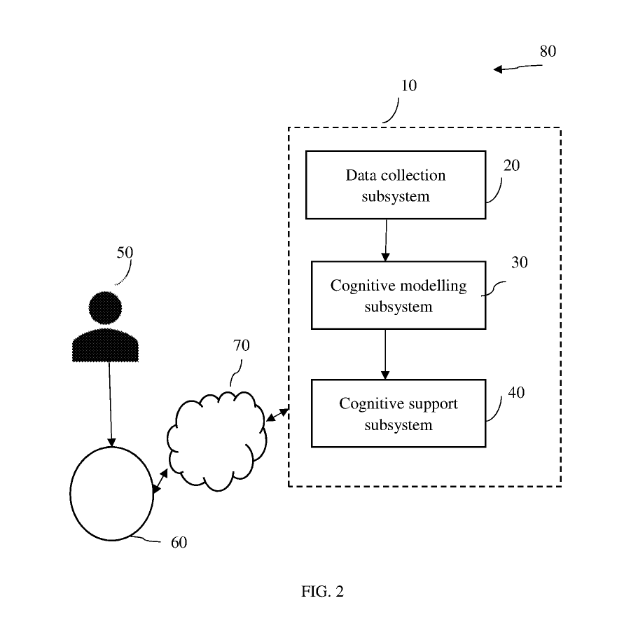

FIG. 2 is a block diagram representation 80 of an exemplary cognitive support system 10 for the sufferer 50 of cognitive dysfunction of FIG. 1 in accordance with an embodiment of the present disclosure. Declines in executive function such as forgetfulness or confusion cause the sufferer 50 to underperform in personal and professional situations. The cognitive support system 10 empowers the sufferer 50 to perform activities of daily living (ADLs), such as managing medications, shopping for groceries, and to continue to perform work-related tasks and to communicate with individuals. When an individual experiences lack of focus, confusion or other cognitive difficulty, the cognitive support system 10 intervenes with contextual cues, enabling the sufferer 50 to prioritize and execute activities as planned.

Considering an example, a person is suffering from Alzheimer's disease. In such case, a data collection subsystem 20 of cognitive support system 10 is configured to obtain personal data and the contextual data related to the sufferer 50 of Alzheimer. The data collection subsystem 20 gathers personal data and contextual data related to the sufferer, from conversations of one or more family members, one or more friends, one or more co-workers and one or more surrounding individuals. The data collection subsystem 20 collects digital footprint including social media profiles, documents, electronic mails and recordings, daily living activities, family composition, social situations, profession history expertise, medical history conditions, entertainment preferences, and data related to spanning the lifetime of the sufferer family history data, chronological family relations, past events and associated time, date and people involved, educational history, past activities at work place and places of living.

A cognitive modelling subsystem 30 of cognitive support system 10 is configured to analyse the personal data and the contextual data related to the sufferer 50 of Alzheimer's. The cognitive modelling subsystem 30 is further configured to build a personalized support model based on an analysed data. After building the personalized support model, a cognitive support subsystem 40 continually updates the personalized model by learning one or more patterns from the personal data and the contextual data related to the sufferer 50 on daily basis. To assist the sufferer 50 of Alzheimer, a monitoring device 60, say a wrist watch, is associated with the sufferer 50. The monitoring device 60 includes a plurality of sensors (not shown in FIG. 2) which is configured to collect data corresponding to the plurality of activities of the sufferer 50. In this example, the system 10 is connected to the wearable device 60 through a communication network 70.

Considering a scenario, where the sufferer 50 of Alzheimer's has a history of forgetfulness. The sufferer 50 left his house to pick his son from the school. After reaching the school, the personalized support model associated with the monitoring device 60 identifies why he has come to school. For that, the cognitive modelling subsystem 30 associated with the monitoring device 60 analyses the personal data and the contextual data to identify the relation of the sufferer 50 with the school. Upon analysing the family history, further analysing the educational history of the sufferer 50 and educational history of sufferer's family members, the personalized support model identifies that sufferer's son attended this school and graduated 17 years ago. After identifying the relation of school with the sufferer 50, the cognitive support subsystem 40 detects the currents situation as an instance of forgetfulness and hence, generates an intervention for the sufferer 50 in the form of audio notification, by saying, “there is no need for you to come here”, your son is not in this school now, he graduated 17 years ago”.

Considering a second scenario, the sufferer 50 of the Alzheimer's used to prepare coffee every day in the morning for his wife but he himself does not like to drink coffee. Last month he lost his wife due to an illness, but some mornings he still prepares the coffee. In the current context, the cognitive support subsystem 40 associated with the monitoring device 60 learned the pattern of the last 32 years and got to know that he should prepare coffee in the morning, and the cognitive support subsystem 40 is also aware from the historic data of the sufferer 50 that he does not like the coffee. In the mornings when he still prepares coffee in the morning, the cognitive support subsystem 60 verifies from the historic data, chronological events and the learnt pattern that he used to prepare the coffee for his wife, but he lost his wife last month. In such situation, the cognitive support subsystem 60 alerts the sufferer by sending a notification that he should not prepare the coffee every day because he does not like coffee and his wife is no more.

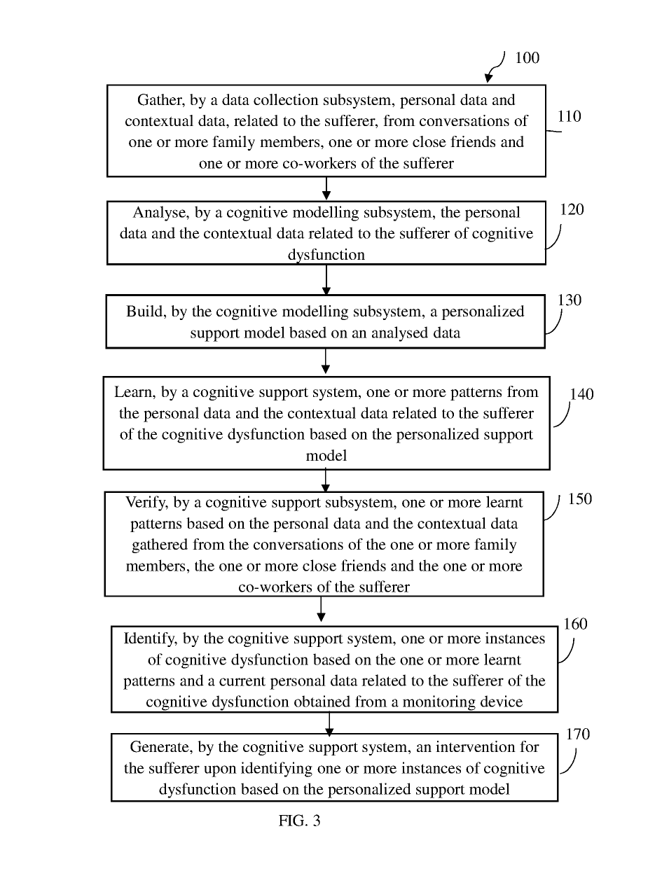

FIG. 3 is a flow chart representing the steps involved in a method 100 for operating the cognitive support system of FIG. 1 in accordance with an embodiment of the present disclosure. The method 100 includes gathering, by a data collection subsystem, personal data and contextual data, related to the sufferer, from conversations of one or more family members, one or more close friends and one or more co-workers of the sufferer in step 110. In one embodiment, gathering personal data related to the sufferer of cognitive dysfunction may include gathering a history of the sufferer and a continuous data collection of the present situation of the sufferer. In such embodiment, gathering the history of the sufferer and the continuous data collection of the present situation of the sufferer may include gathering a heart rate, skin conductance response, skin temperature, posture, motion, voice, reaction to outer stimuli, internal and external location, orientation data, sound, data corresponding to ambient light level, data corresponding to ambient geomagnetic field and proximity data, and environment data based on IoT sensors couple to a plurality of objects in the home. In some embodiments, gathering personal data related to the sufferer of cognitive dysfunction may include gathering digital footprint including at least one of social media profiles, documents, electronic mails and recordings. In a specific embodiment, gathering the contextual data related to the sufferer of cognitive dysfunction may include gathering data about the preferences and background of the user, wherein the data about the preferences and background of the user may include daily living activities, family composition, social situations, professional expertise, medical conditions and entertainment preferences. In another embodiment, gathering contextual data may include gathering a family history data, family relations, chronological events and associated time, date and people involved, educational history, past activities at work place and places of living.

The method 100 also includes analysing, by a cognitive modelling subsystem, the personal data and the contextual data related to the sufferer of cognitive dysfunction in step 120. The method 100 further includes building, by the cognitive modelling subsystem, a personalized support model based on an analysed data in step 130. In some embodiments, building the personalized support model based on the analysed personal data may include building a combination of one or more deep recurrent neural networks models, one or more convolutional neural network models based on the analysed personal data. The method 100 further includes learning, by a cognitive support system, one or more patterns from the personal data and the contextual data related to the sufferer of the cognitive dysfunction based on the personalized support model in step 140. In a specific embodiment, learning the one or more patterns from the personal data and the contextual data related to the sufferer of the cognitive dysfunction based on the personalized support model may include learning at least one of a pattern of repetition, a pattern of omission, or a pattern of abandonment. The method further includes verifying, by a cognitive support subsystem, one or more learnt patterns based on the personal data and the contextual data gathered from the conversations of the one or more family members, the one or more close friends and the one or more co-workers of the sufferer in step 150.

Furthermore, the method 100 includes identifying, by the cognitive support system, one or more instances of cognitive dysfunction based on the one or more learnt patterns and a current personal data related to the sufferer of the cognitive dysfunction obtained from a monitoring device in step 160. In one embodiment, identifying the one or more instances of cognitive dysfunction based on the one or more learnt patterns and a current personal data related to the sufferer of the cognitive dysfunction may include identifying forgetfulness, inability to complete task or change in emotional state of the sufferer based on the one or more learnt patterns and a current personal data related to the sufferer of the cognitive dysfunction.

Moreover, the method 100 further includes generating, by the cognitive support system, an intervention for the sufferer upon identifying one or more instances of cognitive dysfunction based on the personalized support model in step 170. In some embodiments, generating the intervention for the sufferer upon identifying the one or more instances of cognitive dysfunction based on the personalized support model may include generating the intervention for the sufferer based on one or more patterns based personalized support model. In a specific embodiment, generating the intervention for the sufferer upon identifying the one or more instances of cognitive dysfunction may include generating the intervention in the form of one or more notifications. In such embodiment, generating the intervention in the form of one or more notifications may include generating at least one of a notification of context awareness, a notification with one or more instructions to carry out a task and a notification for assistance in speaking. In one embodiment, generating the intervention in the form of one or more notifications may include generating a text notification, an audio notification, a graphical notification or a video notification.

In a specific embodiment, the method 100 may include measuring a performance of the personalized support model based on a generated intervention.

Various embodiments of the cognitive support system described above enables real-time support and interventions to allow individuals affected by cognitive dysfunction to continue to perform activities of daily life. The system computes cognitive function, detects changes in cognitive function, and infers attribution to changes in behavioural activity without disrupting the sufferer's day-to-day activities.

The cognitive support system is highly sensitive, specific, and unobtrusive to the sufferer. The system is helpful in dynamically adapting to situations when a person needs to be reminded to take an action that depends on a previous sequence of actions.

The cognitive support system may be used for the sufferer of cognitive dysfunction to assist the sufferer in daily activities. The cognitive support system may also be used for doctors and researchers for study so that they can easily access the daily activities and other parameters they wish to study. The cognitive support system may be used for caregiver, which often a family member or a medical person, for personalized care of the sufferer.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A cognitive support system for a sufferer of cognitive dysfunction comprising:
a data collection subsystem located on a remote server and configured to:
gather personal data and contextual data, related to the sufferer; from conversations of one or more family members, one or more close friends and one or more co-workers of the sufferer;
a cognitive modelling subsystem operatively coupled to the data collection subsystem and configured to:
analyse the personal data and the contextual data related to the sufferer of cognitive dysfunction;
build a personalized support model based on an analysed data;
a cognitive support subsystem operatively coupled to the cognitive modelling subsystem and configured to:
learn one or more patterns from the personal data and the contextual data related to the sufferer of the cognitive dysfunction based on the personalized support model;
verify one or more learnt patterns based on the personal data and the contextual data gathered from the conversations of the one or more family members, the one or more close friends and the one or more co-workers of the sufferer;
identify one or more instances of cognitive dysfunction based on the one or more learnt patterns and a current personal data related to the sufferer of the cognitive dysfunction obtained from a monitoring device; and
generate an intervention for the sufferer upon identifying one or more instances of cognitive dysfunction based on the personalized support model.

2. The cognitive support system of claim 1, wherein the personal data comprises a historical data of the sufferer and a present situation data of the sufferer.

3. The cognitive support system of claim 2, wherein the historical data of the sufferer and the present situation data of the sufferer comprises a heart rate, skin conductance response, skin temperature, posture, motion, voice, reaction to outer stimuli, internal and external location, orientation data, sound, data corresponding to ambient light level, data corresponding to ambient geomagnetic field and proximity data, and environment data based on IoT sensors couple to a plurality of objects in the home.

4. The cognitive support system of claim 1, wherein the personal data comprises digital footprint comprising at least one of social media profiles, documents, electronic mails and recordings.

5. The cognitive support system of claim 1, wherein the contextual data comprises data about the preferences and background of the user, wherein the data about the preferences and background of the user comprise daily living activities, family composition, social situations, profession history, medical history, entertainment preferences, and data related to spanning the lifetime of the sufferer.

6. The cognitive support system of claim 1, wherein the contextual data comprises a family history data, family relations, chronological events and associated time, date and people involved, educational history, past activities at work place and places of living.

7. The cognitive support system of claim 1, wherein the personalized support model comprises a combination of one or more deep recurrent neural networks models and one or more convolutional neural networks models.

8. The cognitive system of claim 1, wherein the one or more patterns comprises at least one of a pattern of repetition, a pattern of omission, or a pattern of abandonment.

9. The cognitive support system of claim 1, wherein the monitoring device comprises a wearable device.

10. The cognitive support system of claim 1, wherein the intervention comprises one or more notifications.

11. The cognitive support system of claim 8, wherein the one or more notifications comprises at least one of a notification of context awareness, a notification with one or more instructions to carry out a task and one or more notification for assistance in speaking.

12. The cognitive support system of claim 8, wherein the one or more notifications comprises at least one of a text notification, an audio notification, a graphical notification and a video notification.

13. A method comprising:
gathering, by a data collection subsystem, personal data and contextual data, related to the sufferer, from conversations of one or more family members, one or more close friends and one or more co-workers of the sufferer;
analysing, by a cognitive modelling subsystem, the personal data and the contextual data related to the sufferer of cognitive dysfunction;
building, by the cognitive modelling subsystem, a personalized support model and chronological history associated with the sufferer based on an analysed data;
learning, by a cognitive support system, one or more patterns from the personal data and the contextual data related to the sufferer of the cognitive dysfunction based on the personalized support model;
verifying, by a cognitive support subsystem, one or more learnt patterns based on the personal data and the contextual data gathered from the conversations of the one or more family members, the one or more close friends and the one or more co-workers of the sufferer;
identifying, by the cognitive support system, one or more instances of cognitive dysfunction based on the one or more learnt patterns and a current personal data related to the sufferer of the cognitive dysfunction obtained from a monitoring device; and
generating, by the cognitive support system, an intervention for the sufferer upon identifying one or more instances of cognitive dysfunction based on the personalized support model.

14. The method of claim 11, wherein obtaining personal data related to the sufferer of cognitive dysfunction comprises obtaining a history of the sufferer and a continuous data collection of the present situation of the sufferer.

15. The method of claim 11, wherein building the personalized support model based on the analysed personal data comprises building a combination of one or more deep recurrent neural networks models, one or more convolutional neural network models based on the analysed personal data.

16. The method of claim 11, wherein generating the intervention for the sufferer upon identifying the one or more instances of cognitive dysfunction based on the personalized support model comprises generating the intervention for the sufferer based on one or more patterns based personalized support model.

17. The method of claim 11, further comprising measuring a performance of the personalized support model based on a generated intervention.
